(19) United States
(12) Patent Application Publication
(10) Pub. No.: US 2003/0144668 A1
(43) Pub. Date: Jul. 31, 2003

(54) Interference screw made of bone material

(76) Inventors: Matthias Lowel, Nurnberg (DE); Klaus Landis, Heroldsberg (DE); Karl Koschatzky, Erlangen (DE)

Correspondence Address: GIFFORD, KRASS, GROH, SPRINKLE, ANDERSON & CITKOWSKI, PC, 280 N OLD WOODARD AVE, SUITE 400, BIRMINGHAM, MI, 48009, US

(21) Appl. No.: 10/239,618

(22) PCT Filed: Jan. 26, 2001
(86) PCT No.: PCT/EP01/00862

(30) Foreign Application Priority Data
Mar. 24, 2000 (DE) 10014617.1

(51) Int. Cl. A61B 17/56

(52) U.S. Cl. 606/73

(57) ABSTRACT

The invention concerns a rubber composition for making tyres, comprising a diene elastomer, a reinforcing inorganic filler, a polysulphide alkoxysilane as (inorganic filler/elastomer) coupling agent with which are associated an aldimine (of formula R—CH═N—R) and a guanidine derivative. The invention also concerns a tyre or semi-finished product for tyres comprising said rubber composition. The invention further concerns an (inorganic filler/elastomer) coupling system for a rubber composition reinforced with an inorganic filler, consisting of the combination of a polysulphide alkoxysilane, an aldimine (R—CH═N—R) and a guanidine derivative.

The present invention relates to an implant for the fixation of bones, in particular of bone blocks with a tendon portion, especially with an anterior cruciate ligament (ACL) graft.

ACL grafting is a treatment method for patients with a partial or complete rupture of the anterior cruciate ligament in which usually an autologous or allogenic patella bone block/patella tendon bone block (BTB, bone-tendon-bone) is implanted into the knee joint as a cruciate ligament replacement.

The fixation of the implanted bone blocks in the drilled passage of the femur and of the tibia takes place with correspondingly dimensioned interference screws. These consist, for example, of metal (e.g. titanium) or of plastics (e.g. polylactide).

There is, however, a risk with metallic implants of damage to or destruction of the implanted bone blocks, or of the tendon portion, during the implantation. Moreover, these implants remain in the implant bed for life and result in artifact formations in the imaging in diagnostic examinations such as X-rays. A diagnosis of the incorporation success after the operation is thus only possible with limitations.

Implants made of plastics are admittedly absorbed over a period of months, but the monomers or oligomers thereby created can result in damage to the implant or to the implant bed, with corresponding complications such as lysis of the bone.

The object of the present invention therefore consists in providing an implant for the fixation of bone elements or bone blocks which eliminates the above-named disadvantages and provides a reliable fixation in or on the patient bone.

This object is satisfied by an interference screw having the features of claim 1.

A particular advantage of the interference screw in accordance with the invention is provided by the material used which, due to its biological origin, does not represent a foreign body. The implant manufactured of bone material thereby contributes to the fixation and to fusion between the implant and the implant bed in that it is converted into the body's own tissue during incorporation.

The interference screw in accordance with the invention is absorbed over a longer period of time and replaced by the body's own tissue without causing lysis of the implant or of the implant bed by absorption products. The interference screw has a central passage drilling so that it can be introduced into the implant bed in a defined manner with the aid of a corresponding guiding tool. The outer thread hereby ensures a reliable anchoring of the implant in the implant bed without mechanically damaging the implant.

Advantageous embodiments are described in the description, in the drawing and in the dependent claims.

One or more chip flutes, which can be provided at the screw body, can ensure the removal of autologous bone material or tissue material from the implant bed during the screwing in of the interference screw.

Fixation can take place by a suitable application instrument which is applied to an outer square head, or outer hexagon, provided at the screw head. Alternatively, suitable inner openings in the screw with an edged design or as a thread can serve for the reception of an application instrument.

In addition to an application in the femoral and tibial tunnel as part of an ACL graft, an application is also possible in other surgical measures such as the fixation of bone fragments after fractures due to the differently sized dimensions of the interference screw.

In accordance with a preferred embodiment of the invention) the material of the interference screw consists of conserved and sterile bone material of a human or animal origin.

The bone material can consist of spongious, cortical or compact bone or of composites resulting therefrom.

In a particularly advantageous embodiment of the interference screw in accordance with the invention, the screw body has the form of a rotational ellipsoid in the longitudinal direction. Such a design increases the stability of the fixation between the implant and the implant bed.

In accordance with a preferred embodiment of the present invention, the drilling, preferably a passage drilling, is preferably designed such that an implantation into the defined implant bed is possible with the aid of a guiding instrument. The fixation can optionally be controlled during the operation by imaging processes.

In accordance with a further preferred embodiment of the present invention, it is ensured by one or more chip flutes on the screwing in of the interference screw that any bone fragments or tissue fragments which arise are transported out of the implant bed or of the drilling passage and that thus damage to the interference screw or to the implant bed is prevented.

A further preferred embodiment of the interference screw in accordance with the invention has an outer square end, preferably an outer hexagon, at the screw head. In contrast to an inner square end, or inner hexagon, a better and more uniform force transmission from the setting tool to the interference screw is thereby ensured and thus a turning of the screw head off the screw body during the implantation is prevented. Alternatively, suitable inner openings in the screw with an edged design or as a thread can also serve to receive an application instrument.

The interference screw is matched in its size to the drilling passage in the femoral or tibial tunnel, with its diameter generally being approximately 2 mm smaller than the corresponding drilling passage.

The application of the BTB implant and the subsequent fixation with the corresponding interference screws mostly takes place after the setting of the drilling passages into the femur and the tibia by an arthroscopic surgical technique.

A suitable allogenic or xenogenic bone material is processed as the material for the interference screw in accordance with the present invention such that it is conserved, storable and sterile and can be used for the intended purpose. The conservation of the bone material can take place, for example, by freeze drying. However, the bone material is preferably produced by solvent dehydration of collagen bone material by means of an organic solvent miscible with water, e.g. methanol, ethanol, propanol, isopropanol, acetone, methyl ethyl ketone or mixtures of these solvents. The conservation and sterilization of the bone material in accordance with this process is also the subject of the patent DE 29 06 650, whose contents are incorporated by reference into the disclosure of the present application.

This process serves for the manufacture of preserved transplant materials and allows a dehydration and exposure down to the fine structure of the fibril of the collagen bone material such that the processed bone material has a very similar structure to the natural bone in the histological picture and such that the desired properties of the collagen bone material are thus maintained. This process of solvent dehydration moreover has the advantage that a much lower apparatus effort is required in comparison with freeze drying.

Furthermore, the bone material can also be manufactured by solvent dehydration of collagen bone material with a subsequent terminal sterilization, in particular by irradiation with gamma rays or electron rays, but also by ethylene oxide or thermal processes.

Alternatively, the bone material can be produced by aseptic processing of collagen bone material without terminal sterilization.

The present invention will be described in the following purely by way of example with reference to an embodiment and to the enclosed drawings. There are shown:

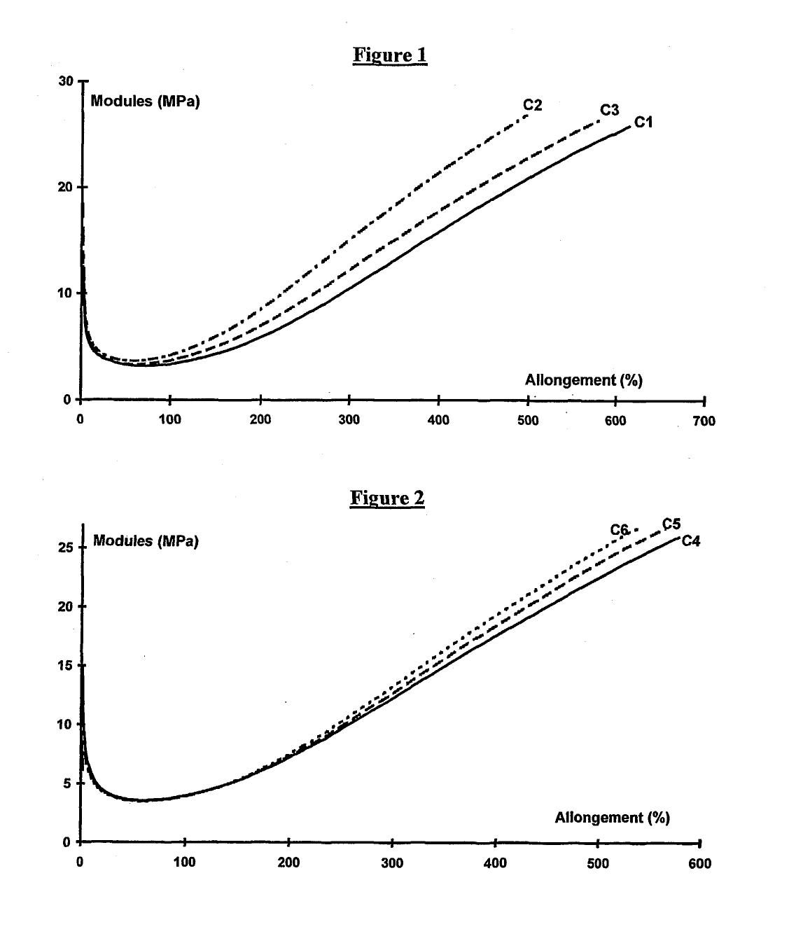

FIG. 1 a partly sectioned side view of an interference screw in accordance with the present invention;

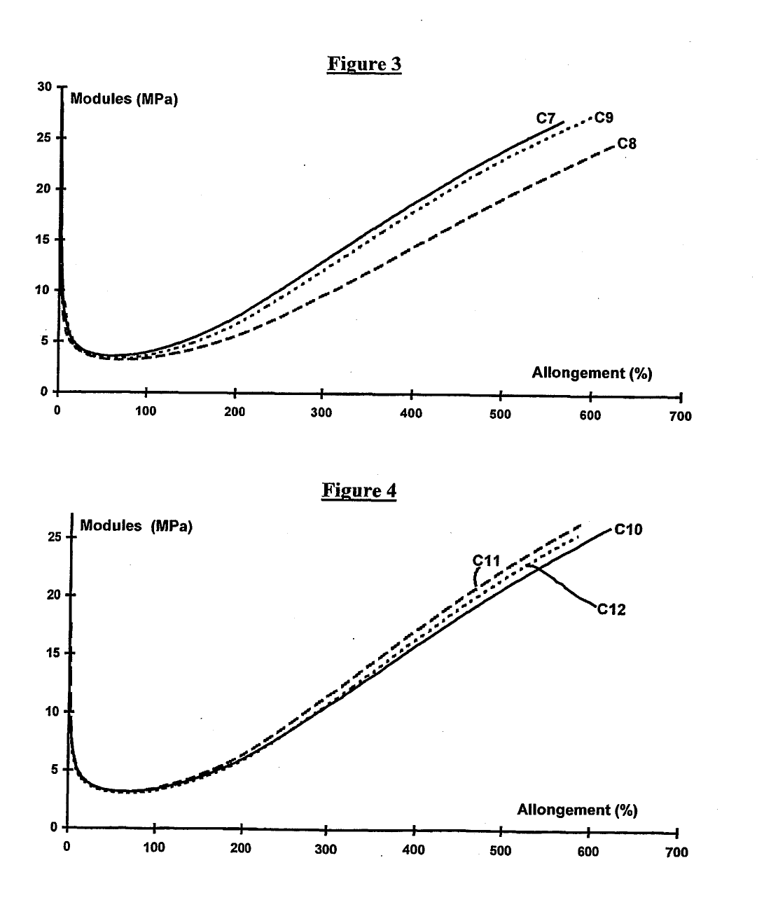

FIG. 2 a side view of the interference screw of FIG. 1 rotated by 90°; and

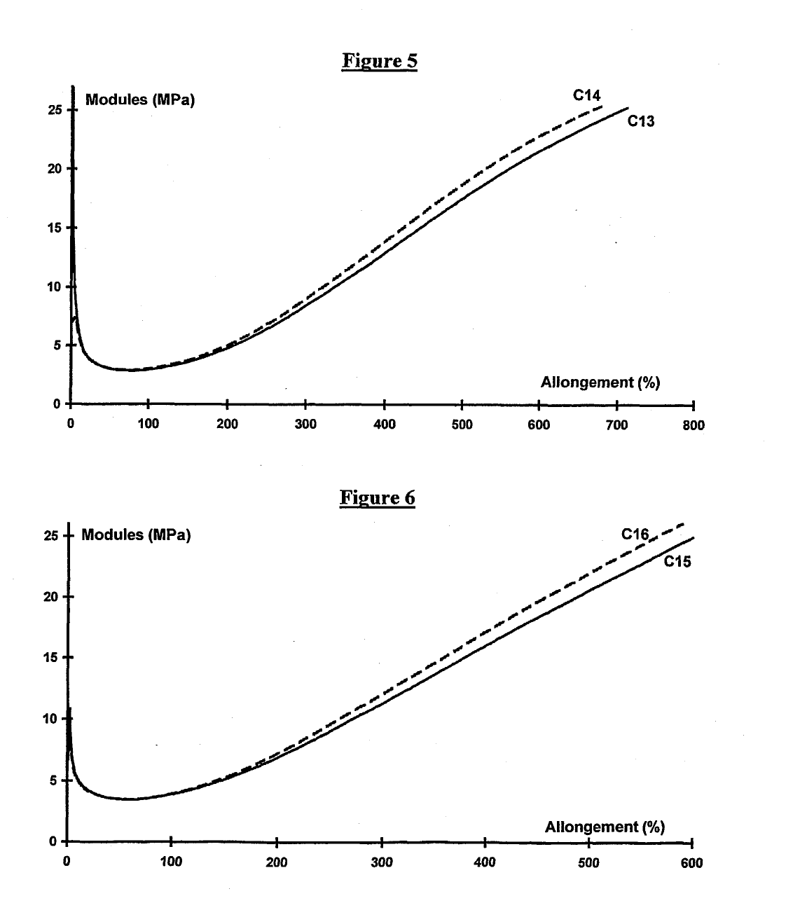

FIG. 3 a plan view of the screw head of the interference screw of FIGS. 1 and 2.

The embodiment shown in FIGS. 1 to 3 of an interference screw in accordance with the invention includes a screw body 1 and a screw head 6 which are made as one piece and which can consist, for example, of compact femoral bone material of e.g. bovine origin.

The screw head 1 has the shape of a rotational ellipsoid and is flattened at its end opposite the screw head 6. A through-hole 2, which runs coaxially to the central axis of the interference screw and extends through the whole interference screw, is provided in the screw body 1 and the screw head 6.

The screw head 6 has an outer hexagon 4 and smaller cross-sectional dimensions than the widest cross-section of the screw body 1.

At the outer periphery of the screw body 1, a self-tapping outer thread 3 is provided which extends over the whole length of the screw body 1. The outer thread 3 is symmetrically interrupted over its whole length by a total of three chip flutes 5 which are uniformly distributed over the periphery of the interference screw.

The screwy body 1 of the interference screw is generally matched to the implant bed in its dimensions. The outer dimensions of such an interference screw can, for example, be as follows depending on the application site in the femoral or in the tibial drilling passage:

| | |
|---|---|
| length (L) | 15 to 30 mm; |
| diameter (D): | 5 to 12 mm; |
| diameter of the | 1 to 3 mm; |
| passage drilling: | |
| outer hexagon: | width over flats 5 to 8 mm. |

The outer thread 3 can be self-tapping or non self-tapping. The thread shape or the module shape is preferably a saw thread. The flank angle preferably amounts to 60°.

The embodiments shown are suitable both for implantation into the femoral and into the tibial drilling passage.

Reference numeral list

| | |
|---|---|
| 1 | screw body |
| 2 | through-hole |
| 3 | outer thread |
| 4 | outer hexagon |
| 5 | chip flutes |
| 6 | screw head |

1. An interference screw consisting of a screw head (6) and a screw body (1) comprising an outer thread (3), with the screw head (6) and the screw body (1) consisting of spongious, cortical or compact bone material and a hole (2) being provided for a guiding instrument in the screw body (1).

2. An interference screw in accordance with claim 1, characterized in that the screw body (1) substantially has the shape of a rotational ellipsoid.

3. An interference screw in accordance with claim 1 or claim 2, characterized in that the screw body (1) has a self-tapping outer thread (3).

4. An interference screw in accordance with any one of the preceding claims, characterized in that the outer thread (3) is interrupted by at least one chip flute (5) which preferably extends over the total axial length of the outer thread (3).

5. An interference screw in accordance with claim 4, characterized in that the cross-section of the chip flute (5) is asymmetrical, V-shaped or rectangular.

6. An interference screw in accordance with any one of the preceding claims, characterized in that the screw head (6) has an outer square end or an outer hexagon (4) or has suitable inner openings with an edged design or a thread to receive an application instrument.

7. An interference screw in accordance with any one of the preceding claims, characterized in that the screw head (6) has smaller outer dimensions than the screw body (1).

8. An interference screw in accordance with claim 1, characterized in that it consists of conserved and sterile bone material of human or animal origin, in particular of conserved and sterile bovine bone material.

9. An interference screw in accordance with any one of the preceding claims, characterized in that the bone material is produced by solvent dehydration of collagen bone material by means of an organic solvent miscible with water, e.g. by means of methanol, ethanol, propanol, isopropanol, acetone, methyl ethyl ketone or mixtures of these solvents.

10. An interference screw in accordance with any one of the preceding claims, characterized in that the bone material is produced by solvent dehydration of collagen bone material with a subsequent terminal sterilization, in particular by irradiation with gamma rays or electron rays.

11. An interference screw in accordance with any one of the preceding claims 1 to 9, characterized in that the bone material is produced by aseptic processing of collagen bone material without terminal sterilizations.
